(19) United States
(12) Patent Application Publication
(10) Pub. No.: US 2025/0251236 A1
(43) Pub. Date: Aug. 7, 2025

(54) STEREOSCOPIC SCANNING DEVICE AND STEREOSCOPIC SCANNING METHOD

(71) Applicant: QISDA CORPORATION, Taoyuan City (TW)

(72) Inventors: Chih-Ming Hu, Taoyuan City (TW); Ying-Fang Lin, Taoyuan City (TW); Cheng-Chieh Juan, New Taipei City (TW)

(73) Assignee: QISDA CORPORATION, Taoyuan City (TW)

(21) Appl. No.: 19/014,144

(22) Filed: Jan. 8, 2025

(30) Foreign Application Priority Data
Feb. 7, 2024 (CN) 202410172420.1

(51) Int. Cl. G01B 11/25; A61B 5/00; A61C 9/00

(52) U.S. Cl. CPC G01B 11/2513; A61B 5/0088; A61C 9/006; G01B 11/2545

(57) ABSTRACT

A stereoscopic scanning device is applied by a stereoscopic scanning method and includes a projection module and an imaging module. The projection module includes a pattern creator, a visible light source and an invisible light source. The pattern creator and the visible light source create a structured light pattern projected onto a target object. The invisible light source emits an invisible light beam to the target object for generating an excitation light beam. The imaging module is disposed adjacent to the projection module and includes a light splitting component, a first optical sensor and a second optical sensor. The light splitting component respectively reflects the structured light pattern and the excitation light beam and allows passing of the structured light pattern and the excitation light beam. The first optical sensor receives light reflected from the light splitting component. The second optical sensor receives light passing through the light splitting component.

### BACKGROUND OF THE INVENTION

### 1. Field of the Invention

The present invention relates to a stereoscopic scanning device and a related stereoscopic scanning method, and more particularly, to a stereoscopic scanning device with dental plaque detection functions and a related stereoscopic scanning method applied to the foresaid stereoscopic scanning device.

### 2. Description of the Prior Art

A conventional oral scanner utilizes the visible light source and the structured light generator to project a structured light pattern onto the teeth, and then utilizes the optical sensor to receive the reflected structured light pattern from the teeth, for analyzing the reflected structured light pattern and acquiring the surface contour of the teeth. The visible light source includes three base colors of the blue light source, the green light source and the red light source. The optical sensor of the conventional oral scanner performs color reconstruction according to the reflection spectrum of the three base color light from the teeth, so that the true color of the surface contour of the teeth are displayed on the display screen connected to the oral scanner. However, the conventional oral scanner only detects the surface contour of the teeth, and design of an oral scanner of detecting the teeth surface contour and the decayed teeth is an important issue in the related medical equipment industry.

### SUMMARY OF THE INVENTION

The present invention provides a stereoscopic scanning device with dental plaque detection functions and a related stereoscopic scanning method applied to the foresaid stereoscopic scanning device for solving above drawbacks.

According to the claimed invention, a stereoscopic scanning device with dental plaque detection functions and is applied to a target object. The stereoscopic scanning device includes a projection module and an imaging module. The projection module includes a structured pattern creator, a visible light source and an invisible light source. The structured pattern creator is adapted to generate a structured light pattern. The visible light source is adapted to emit a visible light beam to the structured pattern creator so that the structured pattern creator is projected onto the target object. The invisible light source is adapted to emit an invisible light beam to the target object for generating at least one excitation light beam. The imaging module is disposed adjacent to the projection module. The imaging module includes a light splitting component, a first optical sensor and a second optical sensor. The light splitting component is adapted to reflect one of the structured light pattern and the excitation light beam, and further to allow passing of the other of the structured light pattern and the excitation light beam. The first optical sensor is adapted to receive light reflected from the light splitting component. The second optical sensor is adapted to receive light passing through the light splitting component. The first optical sensor is a monochromatic light sensor, and the second optical sensor is a monochromatic light sensor or a color light sensor.

According to the claimed invention, the light splitting component is adapted to reflect red light, and allow passing of green light and blue light; the visible light source and the invisible light source are adapted to alternately emit the visible light beam and the invisible light beam at different points of time. Or, the light splitting component is adapted to reflect blue light, and allow passing of green light and red light; the visible light source and the invisible light source are adapted to respectively emit the visible light beam and the invisible light beam at the same point of time, or to alternately emit the visible light beam and the invisible light beam at different points of time.

According to the claimed invention, the stereoscopic scanning device further includes a projection lens assembly disposed on a light end of the structured pattern creator, and the structured light pattern is projected onto the target object through the projection lens assembly. The invisible light beam is projected onto the target object through the projection lens assembly, or is projected onto the target object in a manner that does not pass through the projection lens assembly.

According to the claimed invention, the visible light beam is blue light, the invisible light beam is near ultraviolet light, and the excitation light beam is red fluorescent or green fluorescent. Or, the visible light source includes a blue light emitter, a green light emitter and a red light emitter, and adapted to respectively emit the visible light beam belonging to blue light, green light and red light. An actuation point of time of the invisible light source is different from each actuation point of time of the green light emitter and the red light emitter. The visible light source includes a blue light emitter and a yellow light emitter, or includes a blue light emitter and a white light emitter.

According to the claimed invention, the projection module further includes a color filtering diaphragm disposed on a side of the structured pattern creator facing the target object, the color filtering diaphragm includes a first penetrating region and a second penetrating region, a size of the first penetrating region is smaller than a size of the second penetrating region, the structured light pattern passes through the first penetrating region towards the target object, light beams with other wavelength different from the structured light pattern of the visible light beam and the invisible light beam passes through the second penetrating region towards the target object. The first penetrating region is partly overlapped with or completely overlapped with the second penetrating region. The first penetrating region allows passing of the structured light pattern and the invisible light beam, the second penetrating region blocks the structured light pattern but allows passing of the light beams with other wavelength different from the structured light pattern of the visible light beam and the invisible light beam.

According to the claimed invention, the color filtering diaphragm includes a transparent substrate and a wavelength absorption layer, the transparent substrate is divided into a diaphragm region and an excluded region, the diaphragm region does not have the wavelength absorption layer and is defined as the first penetrating region, the excluded region is covered by the wavelength absorption layer and is defined as the second penetrating region with the diaphragm region. The diaphragm region is a solid structure or a hole structure of the transparent substrate. The diaphragm region is an oval shape, and a long axis direction of the oval shape is parallel to a stripe extending direction of the structured light pattern.

According to the claimed invention, the stereoscopic scanning device further includes an imaging lens assembly disposed on a side of the first optical sensor or the second optical sensor facing the target object, the visible light beam and the invisible light beam pass through the imaging lens assembly. The imaging module further includes an invisible filtering component disposed on a side of the first optical sensor or the second optical sensor facing the target object.

According to the claimed invention, a stereoscopic scanning method includes utilizing a visible light source and a structured pattern creator to project a structured light pattern onto a target object, utilizing an invisible light source to emit an invisible light beam to the target object for generating two excitation light beams, utilizing a first optical sensor and a second optical sensor to respectively receive the two excitation light beams respectively being reflected by a light splitting component and passing through the light splitting component from the target object so as to acquire lesion information of the target object, utilizing the second optical sensor to receive the structured light pattern reflected by the target object and passing through the light splitting component to acquire structure information of the target object, and overlap the lesion information with the structure information to generate a scanning result. The first optical sensor and the second optical sensor are monochromatic light sensors.

The stereoscopic scanning device and the stereoscopic scanning method of the present invention can dispose the color filtering diaphragm on the projection path to increase projection efficiency of the visible light beam and the invisible light beam in the green light band and in the red light band, and further can dispose the first optical sensor and the second optical sensor inside the imaging module in accordance with the property of the light splitting component. If the light splitting component can reflect the red light and allow passing of the green light and the blue light, the first optical sensor and the second optical sensor can be set as the monochromatic light sensor, and the blue light emitter of the visible light source and the invisible light source can alternately emit the visible light beam and the invisible light beam at different points of time. If the light splitting component can reflect the blue light and allow passing of the green light and the red light, the first optical sensor can be set as the monochromatic light sensor and the second optical sensor can be set as the color light sensor, and the blue light emitter of the visible light source and the invisible light source can respectively emit the visible light beam and the invisible light beam at the same point of time, or can alternately emit the visible light beam and the invisible light beam at different points of time. The foresaid embodiments can prevent the green fluorescent and the red fluorescent excited by the invisible light beam projected onto the target object from being affected by the green light and the red light of the visible light beam. Therefore, the stereoscopic scanning device with the dental plaque detection function of the present invention can be switched into the normal mode and the tooth decay mode to respectively provide the structural information of the colorful teeth mode and detect whether the teeth has the lesion information such as the decayed teeth or the structural defect.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a diagram of a stereoscopic scanning device according to an embodiment of the present invention.

FIG. 2 is a side view of a color filtering diaphragm according to the embodiment of the present invention.

FIG. 3 is a diagram of the color filtering diaphragm according to the embodiment of the present invention.

FIG. 4 is a diagram of the color filtering diaphragm according to another embodiment of the present invention.

FIG. 5 is a diagram of a stereoscopic scanning device according to another embodiment of the present invention.

FIG. 6 is a diagram of a stereoscopic scanning device according to another embodiment of the present invention.

FIG. 7 is a flow chart of a stereoscopic scanning method according to the embodiment of the present invention.

FIG. 8 is a flow chart of the stereoscopic scanning method according to the embodiment of the present invention.

### DETAILED DESCRIPTION

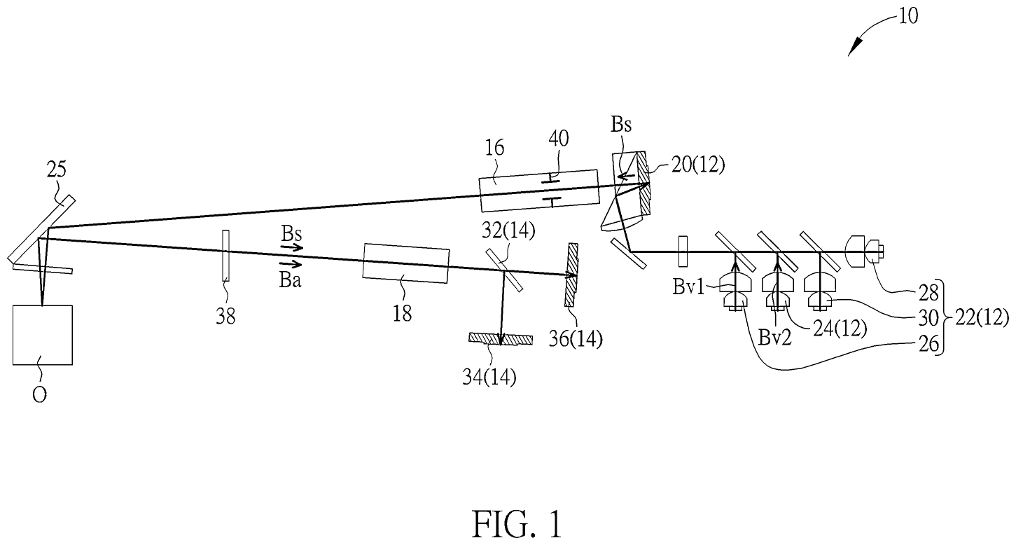

Please refer to FIG. 1. FIG. 1 is a diagram of a stereoscopic scanning device 10 according to an embodiment of the present invention. The stereoscopic scanning device 10 can include a projection module 12, an imaging module 14, a projection lens assembly 16 and an imaging lens assembly 18. The stereoscopic scanning device 10 can be an oral scanner or any related medical equipment. The projection module 12 can be a light source for the oral scanner switched between a normal mode and a tooth decay mode. The imaging module 14 can acquire a scanning result of the stereoscopic scanning device 10 switched in the normal mode and the tooth decay mode. The stereoscopic scanning device 10 switched in the normal mode can rapidly set a teeth model; the stereoscopic scanning device 10 switched in the tooth decay mode can detect whether the teeth is decayed or carious or has any structural defect. The projection lens assembly 16 can be located between the projection module 12 and the target object O. The imaging lens assembly 18 can be located between the imaging module 14 and the target object O. The projection lens assembly 16 and the imaging lens assembly 18 can individually include several optical elements, and a detailed description is omitted herein for simplicity.

The projection module 12 can include a structured pattern creator 20, a visible light source 22 and an invisible light source 24. The structured pattern creator 20 can be a digital micromirror device, or any components with related functions. The visible light source 22 can emit a visible light beam Bv1 towards the structured pattern creator 20, so that the structured pattern creator 20 can generate a structured light pattern Bs that is reflected by a reflector 25 towards the target object O. The visible light source 22 can at least include a blue light emitter 26 used to emit the visible light beam Bv1 belonging to blue light. The visible light source 22 can further include a green light emitter 28 and a red light emitter 30 used to emit the visible light beam Bv1 respectively belonging to green light and red light. A combination of emitters in the visible light source 22 is not limited to the foresaid embodiment, and can be changed due to design demand of an alignment module of the stereoscopic scanning device 10. The invisible light source 24 can emit an invisible light beam Bv2, and the invisible light beam Bv2 can be reflected by the reflector 25 towards the target object O for generating an excitation light beam Ba.

In the present invention, the structured light pattern Bs can belong to a blue light band; the invisible light beam Bv2 can be near ultraviolet light, and a central wavelength of the near ultraviolet light can be about 405 nm, however its application is not limited to the foresaid embodiment. The target object O can be a teeth; if the teeth has bacteria (which means a decayed teeth), the invisible light beam Bv2 can be projected onto the decayed teeth to excite the excitation light beam Ba with red fluorescent; if the teeth has no bacteria, the invisible light beam Bv2 can be projected onto a normal area of the target object O to excite the excitation light beam Ba with green fluorescent. If the teeth is broken or in an early stage of the decayed teeth, the invisible light beam Bv2 can be projected onto the early stage of the teeth to generate a partial black image for the imaging module 14 due to fluorescence of the target object O disappeared, which means some local black area are formed on the teeth model.

The imaging module 14 can be disposed relative to the projection module 12. The imaging module 14 can include a light splitting component 32, a first optical sensor 34 and a second optical sensor 36. The light splitting component 32 can reflect one of the structured light pattern Bs and the excitation light beam Ba, and allow passing of the other of the structured light pattern Bs and the excitation light beam Ba. For example, the light splitting component 32 can reflect the red light, and allow passing of the green light and the blue light; in this embodiment, the first optical sensor 34 and the second optical sensor 36 can be the monochromatic light sensors. The first optical sensor 34 can receive light reflected by the light splitting component 32, such as the red light of the visible light beam Bv1, and the red fluorescent of the excitation light beam Ba; the second optical sensor 36 can receive light passing through the light splitting component 32, such as the structured light pattern Bs, the blue light and the green light of the visible light beam Bv1, and the green fluorescent of the excitation light beam Ba. Therefore, the visible light source 22 and the invisible light source 24 can alternately emit the visible light beam Bv1 and the invisible light beam Bv2 at different points of time, so as to prevent the structured light pattern Bs (that passes through the light splitting component 32 and belongs to the blue light band) and the excitation light beam Ba (that is the green fluorescent) from being interfered with each other.

Or, the light splitting component 32 may reflect the blue light, and allow passing of the green light and the red light. The structured light pattern Bs that belongs to the blue light band can be reflected by the light splitting component 32 and then received by the first optical sensor 34; the red light and the green light of the visible light beam Bv1, and the red fluorescent and the green fluorescent of the excitation light beam Ba can pass through the light splitting component 32 to be received by the second optical sensor 36, so that the first optical sensor 34 can be designed as the monochromatic light sensor and the second optical sensor 36 can be designed as the color light sensor. Thus, the visible light source 22 and the invisible light source 24 can respectively emit the visible light beam Bv1 and the invisible light beam Bv2 that belong to the blue light at the same point of time, or can alternately emit the visible light beam Bv1 and the invisible light beam Bv2 that belong to the blue light at different points of time; an actuation point of time of the invisible light source 24 can be preferably different from each actuation point of time of the green light emitter 28 and the red light emitter 30 of the visible light source 22 to avoid interference.

Besides, the projection lens assembly 16 can be located on a light end of the structured pattern creator 20, and the structured light pattern Bs can pass through the projection lens assembly 16 to be projected onto the target object O; in this embodiment, the invisible light beam Bv2 can pass through the projection lens assembly 16 to be projected onto the target object O. The imaging lens assembly 18 can be disposed on a side of the first optical sensor 34 and/or the second optical sensor 36 facing the target object O. The visible light beam Bv1 and the invisible light beam Bv2 can reflected by the target object O and pass through the imaging lens assembly 18 for being received by the first optical sensor 34 and/or the second optical sensor 36 via the light splitting component 32. Moreover, the imaging lens assembly 18 can optionally include an invisible filtering component 38 disposed on the side of the first optical sensor 34 and/or the second optical sensor 36 that faces the target object O. When the stereoscopic scanning device 10 is switched into the tooth decay mode and the projection module 12 utilizes the invisible light source 24 to emit the invisible light beam Bv2, the invisible light beam Bv2 projected onto the target object O (such as the teeth) can be excited to generate the excitation light beam Ba, and some of the invisible light beam Bv2 may be reflected by the target object O to enter an imaging path, so that the invisible filtering component 38 can be used to filter the invisible light beam Bv2 reflected by the target object O, so as to prevent a detection result of the tooth decay mode from being affected.

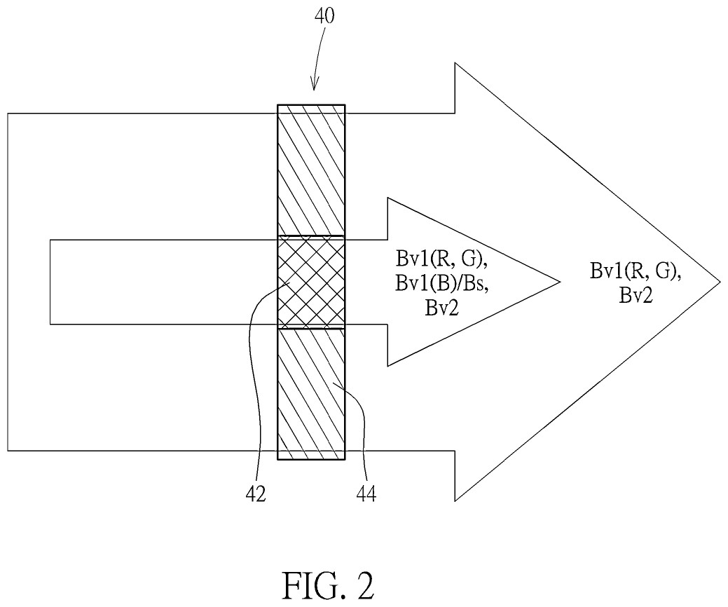

The projection module 12 can further include a color filtering diaphragm 40 disposed on a side of the structured pattern creator 20 facing the target object O. Please refer to FIG. 1 and FIG. 2. FIG. 2 is a side view of a color filtering diaphragm 40 according to the embodiment of the present invention. The color filtering diaphragm 40 can include a first penetrating region 42 and a second penetrating region 44; the first penetrating region 42 can be represented by a plaid area, and the second penetrating region 44 can be represented by a twill area covering the plaid area, so that a size of the first penetrating region 42 can be smaller than a size of the second penetrating region 44, and the first penetrating region 42 can be overlapped with the second penetrating region 44 partly or completely. The first penetrating region 42 can allow passing of the visible light beam Bv1 (such as the green light and the red light and the structured light pattern Bs) and all color light of the invisible light beam Bv2; the second penetrating region 44 can block the blue light (such as a band range of the structured light pattern Bs), but allow passing of the color light with other wavelengths. Therefore, in a projection path from the structured pattern creator 20 to the reflector 25, the structured light pattern Bs generated by the structured pattern creator 20 can belong to the blue light band, and only can pass through the first penetrating region 42 towards the target object O; the visible light beam Bv1 with other wavelengths that do not belong to the structured light pattern Bs and the invisible light beam Bv2 can pass through the second penetrating region 44 to arrive the target object O.

The blue light emitter 26 can be set on position close to the structured pattern creator 20, and the green light emitter 28 and the red light emitter 30 with longer band ranges can be set on position distant from the structured pattern creator 20 and has decreased projection efficiency. Thus, the present invention can design the color filtering diaphragm 40 with two diaphragms of different sizes. The first penetrating region 42 of the color filtering diaphragm 40 can be defined as a small diaphragm, all light that includes the structured light pattern Bs can pass through the first penetrating region 42 to keep the depth of field of the detection image; the second penetrating region 44 of the color filtering diaphragm 40 can be defined as a large diaphragm, which is used to block the structured light pattern Bs and allow passing of the invisible light beam Bv2 and the visible light beam Bv1 with other base color (such as the green light and the red light), so as to increase the projection efficiency of the visible light beam Bv1 with other wavelengths that do not belong to the structured light pattern Bs and the projection efficiency of the invisible light beam Bv2.

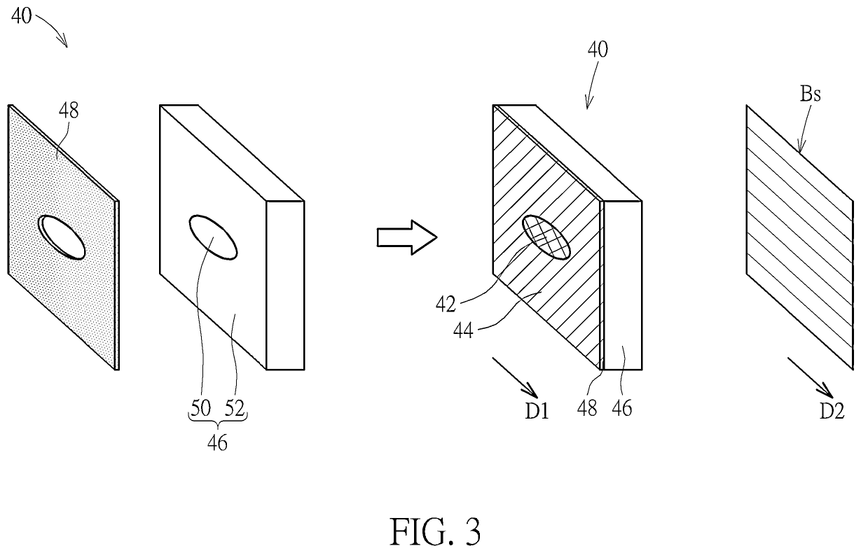

Please refer to FIG. 2 and FIG. 3. FIG. 3 is a diagram of the color filtering diaphragm 40 according to the embodiment of the present invention. The color filtering diaphragm 40 can include a transparent substrate 46 and a wavelength absorption layer 48, and the transparent substrate 46 can include a diaphragm region 50 and an excluded region 52. The wavelength absorption layer 48 can block the structured light pattern Bs, but allow passing of the invisible light beam Bv2 and the visible light beam Bv1 with other wavelengths that do not belong to the structured light pattern Bs. The diaphragm region 50 is not covered by the wavelength absorption layer 48, and can be indicated as the first penetrating region 42 of the color filtering diaphragm 40. The excluded region 52 can be covered by the wavelength absorption layer 48, and the excluded region 52 and the diaphragm region 50 can be indicated as the second penetrating region 44 of the color filtering diaphragm 40. The diaphragm region 50 can be preferably designed as an oval shape, and a long axis direction D1 of the oval shape can be substantially parallel to a stripe extending direction D2 of the structured light pattern Bs, for preferred effect of the depth of field.

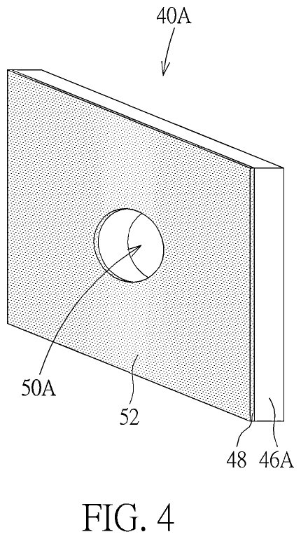

The embodiment shown in FIG. 3 can set a specific region on the transparent substrate 46 as the diaphragm region 50, and other region of the transparent substrate 46 other than the specific region can be set as the excluded region 52, which means the diaphragm region 50 can be a solid structure of the transparent substrate 46, and practical application of the diaphragm region 50 is not limited to the foresaid embodiment. Please refer to FIG. 4. FIG. 4 is a diagram of the color filtering diaphragm 40A according to another embodiment of the present invention. In this embodiment, elements having the same numerals as ones of the foresaid embodiment can have the same structures and functions, and the detailed description is omitted herein for simplicity. The diaphragm region 50A of the color filtering diaphragm 40A can be designed as a hole structure of the transparent substrate 46A. The diaphragm region 50A can be defined as the first penetrating region 42 of the color filtering diaphragm 40A and cannot be covered by the wavelength absorption layer 48. The excluded region 52 of the color filtering diaphragm 40A can be covered by the wavelength absorption layer 48, and the excluded region 52 and the diaphragm region 50A can be defined as the second penetrating region 44 of the color filtering diaphragm 40A.

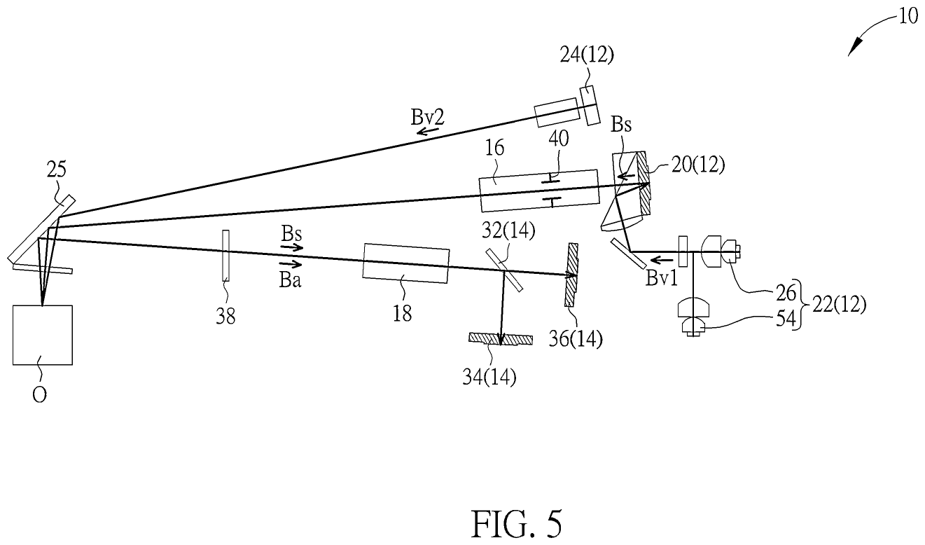

Please refer to FIG. 5. FIG. 5 is a diagram of a stereoscopic scanning device 10 according to another embodiment of the present invention. In this embodiment, elements having the same numerals as ones of the foresaid embodiment can have the same structures and functions, and the detailed description is omitted herein for simplicity. The visible light source 22 of the stereoscopic scanning device 10 can include the blue light emitter 26 and a yellow light emitter 54; the yellow light can be decomposed into the green light and the red light, so that a filtering component (which is not marked in the figures) can be optionally disposed on the projection path of the yellow light emitted by the yellow light emitter 54, or the yellow light detection image may be directly captured without the filtering component; the first optical sensor 34 and/or the second optical sensor 36 can receive the three base color light to acquire the colorful detection image. In addition, the invisible light source 24 can be disposed on position outside the projection path of the projection lens assembly 16, and the invisible light beam Bv2 emitted by the invisible light source 24 can be projected onto the target object O without passing through the projection lens assembly 16.

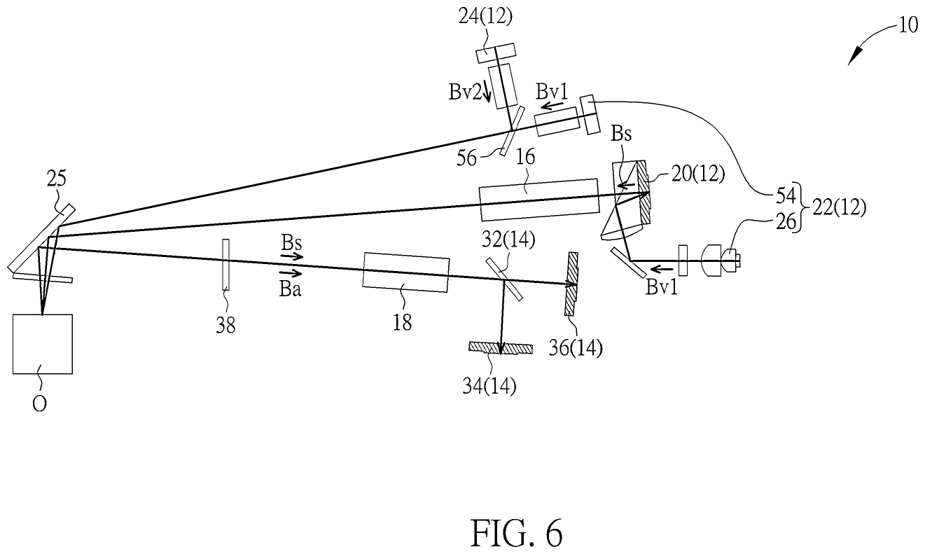

In other possible embodiment, the emitter of the visible light source 22 other than the blue light emitter 26 can be moved out of the alignment module of the blue light emitter 26. Please refer to FIG. 6. FIG. 6 is a diagram of a stereoscopic scanning device 10 according to another embodiment of the present invention. In this embodiment, elements having the same numerals as ones of the foresaid embodiment can have the same structures and functions, and the detailed description is omitted herein for simplicity. In the embodiment, the visible light source 22 of the stereoscopic scanning device 10 can include a blue light emitter 26 and a yellow light emitter 54, and the yellow light emitter 54 can be disposed outside the projection path of the projection lens assembly 16; for example, the yellow light emitter 54 can be disposed near by the invisible light source 24, and a light splitting component 56 can be disposed relative to the invisible light source 24 and the yellow light emitter 54 accordingly. In the embodiment, the projection module 12 does not need to have the color filtering diaphragm. It should be mentioned that the yellow light emitter 54 may be replaced by the white light emitter (which is not marked in the figures), and practical application of the yellow light emitter or the white light emitter can depend on the design demand.

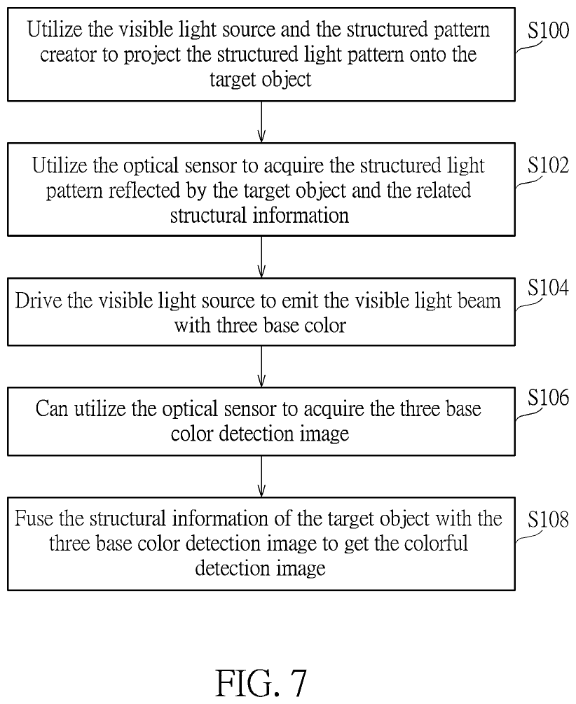

Please refer to FIG. 7. FIG. 7 is a flow chart of a stereoscopic scanning method according to the embodiment of the present invention. When the stereoscopic scanning device 10 is switched into the normal mode, step S100 can utilize the blue light of the visible light source 22 and the structured pattern creator 20 to project the structured light pattern Bs towards the target object O. The structured light pattern Bs can pass through the first penetrating region 42 of the color filtering diaphragm 40, and be blocked by the second penetrating region 44 of the color filtering diaphragm 40. Then, according to the property of the light splitting component 32 (for example, the red light can be reflected and the green light and the blue light can pass by, or the blue light can be reflected and the green light and the red light can pass by), step S102 can utilize the first optical sensor 34 or the second optical sensor 36 to acquire the structured light pattern Bs that is reflected by the target object O and passes through the imaging lens assembly 18, so as to acquire structural information of the target object O. Then, step S104 can drive the visible light source 22 to emit the visible light beam Bv1 with three base color band; the green light and the red light can pass through the second penetrating region 44 of the color filtering diaphragm 40. Step S106 can utilize the first optical sensor 34 or the second optical sensor 36 to acquire the blue light detection image, the green light detection image and the red light detection image of the target object O. Final, step S108 can fuse the structural information of the target object O with the blue light detection image, the green light detection image and the red light detection image for getting the final colorful detection image.

The foresaid stereoscopic scanning method can be applied to the stereoscopic scanning device 10 shown in FIG. 1, and practical application of the stereoscopic scanning method is not limited to this embodiment. For example, the stereoscopic scanning method can be further applied to the stereoscopic scanning device 10 shown in FIG. 5 and FIG. 6; in the embodiment, step S104, step S106 and step S108 can drive the blue light emitter 26 and the yellow light emitter 54 of the visible light source 22 to respectively emit the blue light and the yellow light, and then the first optical sensor 34 or the second optical sensor 36 can acquire the blue light detection image and the yellow light detection image of the target object O, and final the structural information of the target object O can be fused with the blue light detection image and the yellow light detection image to generate the colorful detection image. Or, the stereoscopic scanning device 10 shown in FIG. 6 can replace the yellow light emitter 54 by the white light emitter, and the stereoscopic scanning method can acquire the structural information of the target object O in step S102, and then step S104 and step S106 can drive the white light emitter to emit the white light, and the white light detection image acquired by the first optical sensor 34 or the second optical sensor 36 can be fused with the structural information of the target object O to generate the colorful detection image.

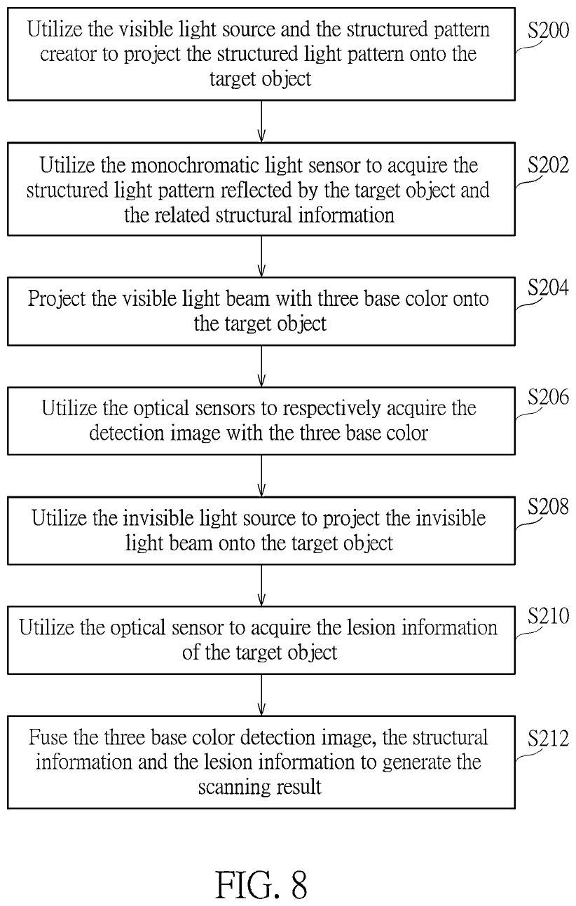

Please refer to FIG. 8. FIG. 8 is a flow chart of the stereoscopic scanning method according to the embodiment of the present invention. The stereoscopic scanning method in the tooth decay mode can take an example of the stereoscopic scanning device 10 shown in FIG. 1, and practical application of the stereoscopic scanning method in the tooth decay mode is not limited to the embodiment. When the stereoscopic scanning device 10 is switched into the tooth decay mode, step S200 can be executed to utilize the blue light of the visible light source 22 and the structured pattern creator 20 to project the blue light pattern of the structured light pattern Bs onto the target object O; the structured light pattern Bs can pass through the first penetrating region 42 of the color filtering diaphragm 40 to arrive the target object O, and be blocked by the second penetrating region 44 of the color filtering diaphragm 40. Then, step S202 can be executed to utilize the first optical sensor 34 or the second optical sensor 36 to receive the structured light pattern Bs reflected by the target object O for acquiring the structural information of the target object O. In step S202, if the light splitting component 32 can reflect the red light and allow passing of the green light and the blue light, the second optical sensor 36 that is the monochromatic light sensor can be used to acquire the structured light pattern Bs; if the light splitting component 32 can reflect the blue light and allow passing of the green light and the red light, the first optical sensor 34 that is the monochromatic light sensor can be used to acquire the structured light pattern Bs.

Then, step S204 and step S206 can be optionally executed to project the visible light beam Bv1 of the visible light source 22 that contains the three base color band onto the target object O, and utilize the first optical sensor 34 and the second optical sensor 36 to respectively acquire the detection image with the three base color. In step S204, if the light splitting component 32 can reflect the red light and allow passing of the green light and the blue light, the first optical sensor 34 and the second optical sensor 36 that are the monochromatic light sensor can respectively acquire the red light detection image and the green light detection image; if the light splitting component 32 can reflect the blue light and allow passing of the green light and the red light, the second optical sensor 36 that is the color light sensor can directly acquire the red light and green light detection image. Besides, step S204 can decide whether to emit the visible light beam Bv1 containing the blue light and the red light and the green light, or the visible light beam Bv1 containing the blue light and the yellow light, or the visible light beam Bv1 containing the white light in accordance with an emitter combination of the visible light source 22; practical application of the visible light beam Bv1 can depend on light design of the stereoscopic scanning device 10.

Then, step S208 can be executed to utilize the invisible light source 24 to project the invisible light beam Bv2 onto the target object O; in the meantime, the invisible light beam Bv2 can pass through the second penetrating region 44 of the color filtering diaphragm 40. Final, step S210 and step S212 can be executed to utilize the first optical sensor 34 or the second optical sensor 36 to receive the excitation light beam Ba generated by the target object O for getting lesion information of the target object O, and then the three base color detection image, the structural information and the lesion information can be fused to generate the scanning result. In step S210, if the light splitting component 32 can reflect the red light and allow passing of the green light and the blue light, the first optical sensor 34 and the second optical sensor 36 that are the monochromatic light sensor can receive the red fluorescent and the green fluorescent of the excitation light beam Ba respectively; if the light splitting component 32 can reflect the blue light and allow passing of the green light and the red light, the second optical sensor 36 that is the color light sensor can directly receive the red fluorescent and the green fluorescent of the excitation light beam Ba. The invisible filtering component 38 and the color filtering diaphragm 40 in the present invention are optional elements, and the foresaid flow chart does not explain its application in detail.

In conclusion, the stereoscopic scanning device and the stereoscopic scanning method of the present invention can dispose the color filtering diaphragm on the projection path to increase projection efficiency of the visible light beam and the invisible light beam in the green light band and in the red light band, and further can dispose the first optical sensor and the second optical sensor inside the imaging module in accordance with the property of the light splitting component. If the light splitting component can reflect the red light and allow passing of the green light and the blue light, the first optical sensor and the second optical sensor can be set as the monochromatic light sensor, and the blue light emitter of the visible light source and the invisible light source can alternately emit the visible light beam and the invisible light beam at different points of time. If the light splitting component can reflect the blue light and allow passing of the green light and the red light, the first optical sensor can be set as the monochromatic light sensor and the second optical sensor can be set as the color light sensor, and the blue light emitter of the visible light source and the invisible light source can respectively emit the visible light beam and the invisible light beam at the same point of time, or can alternately emit the visible light beam and the invisible light beam at different points of time. The foresaid embodiments can prevent the green fluorescent and the red fluorescent excited by the invisible light beam projected onto the target object from being affected by the green light and the red light of the visible light beam. Therefore, the stereoscopic scanning device with the dental plaque detection function of the present invention can be switched into the normal mode and the tooth decay mode to respectively provide the structural information of the colorful teeth mode and detect whether the teeth has the lesion information such as the decayed teeth or the structural defect.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A stereoscopic scanning device with dental plaque detection functions and applied to a target object, the stereoscopic scanning device comprising:
a projection module, comprising:
a structured pattern creator adapted to generate a structured light pattern;
a visible light source adapted to emit a visible light beam to the structured pattern creator so that the structured pattern creator is projected onto the target object; and
an invisible light source adapted to emit an invisible light beam to the target object for generating at least one excitation light beam; and
an imaging module disposed adjacent to the projection module, comprising:
a light splitting component adapted to reflect one of the structured light pattern and the excitation light beam, and further to allow passing of the other of the structured light pattern and the excitation light beam;
a first optical sensor adapted to receive light reflected from the light splitting component; and
a second optical sensor adapted to receive light passing through the light splitting component;
wherein the first optical sensor is a monochromatic light sensor, the second optical sensor is a monochromatic light sensor or a color light sensor.

2. The stereoscopic scanning device of claim 1, wherein the light splitting component is adapted to reflect red light, and allow passing of green light and blue light.

3. The stereoscopic scanning device of claim 2, wherein the visible light source and the invisible light source are adapted to alternately emit the visible light beam and the invisible light beam at different points of time.

4. The stereoscopic scanning device of claim 1, wherein the light splitting component is adapted to reflect blue light, and allow passing of green light and red light.

5. The stereoscopic scanning device of claim 4, wherein the visible light source and the invisible light source are adapted to respectively emit the visible light beam and the invisible light beam at the same point of time, or to alternately emit the visible light beam and the invisible light beam at different points of time.

6. The stereoscopic scanning device of claim 1, wherein the stereoscopic scanning device further comprises a projection lens assembly disposed on a light end of the structured pattern creator, the structured light pattern is projected onto the target object through the projection lens assembly.

7. The stereoscopic scanning device of claim 6, wherein the invisible light beam is projected onto the target object through the projection lens assembly, or is projected onto the target object in a manner that does not pass through the projection lens assembly.

8. The stereoscopic scanning device of claim 1, wherein the visible light beam is blue light, the invisible light beam is near ultraviolet light, the excitation light beam is red fluorescent or green fluorescent.

9. The stereoscopic scanning device of claim 1, wherein the visible light source comprises a blue light emitter, a green light emitter and a red light emitter, and adapted to respectively emit the visible light beam belonging to blue light, green light and red light.

10. The stereoscopic scanning device of claim 9, wherein an actuation point of time of the invisible light source is different from each actuation point of time of the green light emitter and the red light emitter.

11. The stereoscopic scanning device of claim 1, wherein the visible light source comprises a blue light emitter and a yellow light emitter, or comprises a blue light emitter and a white light emitter.

12. The stereoscopic scanning device of claim 1, wherein the projection module further comprises a color filtering diaphragm disposed on a side of the structured pattern creator facing the target object, the color filtering diaphragm comprises a first penetrating region and a second penetrating region, a size of the first penetrating region is smaller than a size of the second penetrating region, the structured light pattern passes through the first penetrating region towards the target object, light beams with other wavelength different from the structured light pattern of the visible light beam and the invisible light beam passes through the second penetrating region towards the target object.

13. The stereoscopic scanning device of claim 12, wherein the first penetrating region is partly overlapped with or completely overlapped with the second penetrating region.

14. The stereoscopic scanning device of claim 12, wherein the first penetrating region allows passing of the structured light pattern and the invisible light beam, the second penetrating region blocks the structured light pattern but allows passing of the light beams with other wavelength different from the structured light pattern of the visible light beam and the invisible light beam.

15. The stereoscopic scanning device of claim 12, wherein the color filtering diaphragm comprises a transparent substrate and a wavelength absorption layer, the transparent substrate is divided into a diaphragm region and an excluded region, the diaphragm region does not have the wavelength absorption layer and is defined as the first penetrating region, the excluded region is covered by the wavelength absorption layer and is defined as the second penetrating region with the diaphragm region.

16. The stereoscopic scanning device of claim 15, wherein the diaphragm region is a solid structure or a hole structure of the transparent substrate.

17. The stereoscopic scanning device of claim 15, wherein the diaphragm region is an oval shape, and a long axis direction of the oval shape is parallel to a stripe extending direction of the structured light pattern.

18. The stereoscopic scanning device of claim 1, wherein the stereoscopic scanning device further comprises an imaging lens assembly disposed on a side of the first optical sensor or the second optical sensor facing the target object, the visible light beam and the invisible light beam pass through the imaging lens assembly.

19. The stereoscopic scanning device of claim 1, wherein the imaging module further comprises an invisible filtering component disposed on a side of the first optical sensor or the second optical sensor facing the target object.

20. A stereoscopic scanning method, comprising:
utilizing a visible light source and a structured pattern creator to project a structured light pattern onto a target object;
utilizing an invisible light source to emit an invisible light beam to the target object for generating two excitation light beams;
utilizing a first optical sensor and a second optical sensor to respectively receive the two excitation light beams respectively being reflected by a light splitting component and passing through the light splitting component from the target object so as to acquire lesion information of the target object;
utilizing the second optical sensor to receive the structured light pattern reflected by the target object and passing through the light splitting component to acquire structure information of the target object; and
overlap the lesion information with the structure information to generate a scanning result;
wherein the first optical sensor and the second optical sensor are monochromatic light sensors.

21. The stereoscopic scanning method of claim 20, wherein the light splitting component is adapted to reflect red light, and allow passing of green light and blue light, the visible light source and the invisible light source are adapted to alternately emit the visible light beam and the invisible light beam at different points of time.

22. A stereoscopic scanning method, comprising:
utilizing a visible light source and a structured pattern creator to project a structured light pattern onto a target object;
utilizing an invisible light source to emit an invisible light beam to the target object for generating two excitation light beams;
utilizing a first optical sensor to receive the structured light pattern reflected by the target object and a light splitting component so as to acquire structure information of the target object;
utilizing a second optical sensor to receive the two excitation light beams excited by the target object and passing through the light splitting component so as to acquire lesion information of the target object; and
overlap the lesion information with the structure information to generate a scanning result;
wherein the first optical sensor is a monochromatic light sensor, and the second optical sensor is a monochromatic light sensor or a color light sensor.

23. The stereoscopic scanning method of claim 22, wherein the light splitting component is adapted to reflect blue light, and allow passing of green light and red light, the visible light source and the invisible light source are adapted to respectively emit the visible light beam and the invisible light beam at the same point of time, or to alternately emit the visible light beam and the invisible light beam at different points of time.
